(19) United States
(12) Patent Application Publication
(10) Pub. No.: US 2023/0053875 A1
(43) Pub. Date: Feb. 23, 2023

(54) MEDICAL DEVICE WITH ANGLED HEADER AND DELIVERY SYSTEM

(71) Applicant: Medtronic, Inc., Minneapolis, MN (US)

(72) Inventors: Kaileigh E. Rock, Mahtomedi, MN (US); Michael D. Eggen, Chisago City, MN (US); Jason D. Hamack, Ramsey, MN (US)

(21) Appl. No.: 17/813,281

(22) Filed: Jul. 18, 2022

Related U.S. Application Data
(60) Provisional application No. 63/234,347, filed on Aug. 18, 2021.

(51) Int. Cl. A61N 1/05; A61N 1/362; A61N 1/375

(52) U.S. Cl. CPC A61N 1/0565; A61N 1/057; A61N 1/3622; A61N 1/37518; A61N 1/0573; A61N 2001/058

(57) ABSTRACT

In some examples, an implantable medical device (IMD) includes a housing defined in part by a longitudinal axis, a header at a distal end of the housing, the header defining a header plane, the header plane disposed at an angle relative to a reference plane, where the reference plane is perpendicular to the longitudinal axis, a fixation mechanism extending from the header, the fixation mechanism configured to be advanced into tissue of a heart of a patient to fix at least a portion of the IMD to the heart, one or more electrodes that extend from the header and are configured to engage with the septum, and circuitry within the housing, wherein the circuitry is configured to deliver cardiac pacing to the heart via the one or more electrodes.

This application claims the benefit of U.S. Provisional Patent Application Ser. No. 63/234,347, filed Aug. 18, 2021, the entire content of which is incorporated herein by reference.

### TECHNICAL FIELD

This disclosure relates generally to medical devices, and, more particularly, to medical devices and systems for delivering medical devices.

### BACKGROUND

Some types of implantable medical devices (IMDs), such as cardiac pacemakers or implantable cardioverter defibrillators systems, may be used to provide cardiac sensing and therapy for a patient via one or more electrodes. Some IMDs include an implantable pulse generator that includes a housing that encloses electronic components, which may be configured to be implanted subcutaneously in the chest of the patient or within a chamber of a heart of the patient, as examples. IMDs having a pulse generator that is configured to be implanted within a chamber of the heart may be referred to as an intracardiac device or a leadless implantable medical device. A medical device delivery system including a delivery catheter may be used to deliver an intracardiac device transvenously to an implant site within a heart of a patient and release the device after the device has been fixed at the implant site. The medical device delivery system then may be withdrawn from the patient.

### SUMMARY

In general, this disclosure is directed to examples of implantable medical devices (IMDs), such as intracardiac devices having a header configured for placement within certain portions of the heart that may be difficult to reach, where the header comprises the distal end portion of the IMD. For instance, header geometry may be configured to allow a reduction of a curve on the delivery catheter coming out of the Inferior Vena Cava and coming over the Eustacian Ridge in a right atrium of a heart, while still providing effective fixation of the IMD to tissue of the triangle of Koch (TOK) or other implant site. In some examples, the header of the implantable medical device may be disposed at an angle so that the device itself is not perpendicular to the implant site.

In one example, an implantable medical device (IMD) includes a housing defined in part by a longitudinal axis, a header at a distal end of the housing, the header defining a header plane, the header plane disposed at an angle relative to a reference plane, where the reference plane is perpendicular to the longitudinal axis, a fixation mechanism extending from the header, the fixation mechanism configured to be advanced into tissue of a heart of a patient to fix at least a portion of the IMD to the heart, one or more electrodes that extend from the header and are configured to engage with the septum, and circuitry within the housing, wherein the circuitry is configured to deliver cardiac pacing to the heart via the one or more electrodes.

In another example, a medical device system comprises an implantable medical device (IMD) having a housing defined in part by a longitudinal axis, a header at a distal end of the housing, the header defining a header plane, the header plane disposed at an angle relative to a reference plane, where the reference plane is perpendicular to the longitudinal axis, a fixation mechanism extending from the header, the fixation mechanism configured to be advanced into tissue of a heart of a patient to fix at least a portion of the IMD to the heart, one or more electrodes that extend from the header and are configured to engage with the septum, and circuitry within the housing, wherein circuitry is configured to deliver cardiac pacing to the heart via the one or more electrodes, and a catheter extending to a distal end, the catheter having a cup at the distal end of the catheter, the cup having a distal cup end, the cup end defined by a cup end plane and a cup longitudinal axis, and the IMD is configured to be disposed within the cup while the IMD is delivered to the septum within the patient.

In another example, a method for using a medical device system comprises advancing a medical device system within a patient, the medical device system comprising a catheter extending to a distal end, the catheter having a cup at the distal end of the catheter, the cup having a distal cup end, the cup end defined by a cup end plane and a cup longitudinal axis, the cup end plane disposed at a cup angle relative to the cup longitudinal axis, an implantable medical device (IMD) disposed within the cup, the IMD comprising a housing having a header, the header having one or more electrodes, the housing defined in part by a longitudinal axis, the header is defined by a header plane, the header plane disposed at an angle relative to a reference plane, where the reference plane is perpendicular to the longitudinal axis, the IMD comprising a plurality of tines configured to be advanced into a septum of a heart of a patient, delivering the IMD to an implant site with the catheter and placing the cup end plane on the tissue where the cup longitudinal axis is non-orthogonal relative to a tissue plane, advancing the IMD from the cup, engaging the plurality of tines with the tissue.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF THE DRAWINGS

FIG. 1 is a conceptual drawing illustrating portions of patient anatomy including potential implant sites for an implantable medical device (IMD);

FIG. 2A is a conceptual drawing illustrating an example implantable medical device (IMD);

FIG. 2B is a plan view illustrating an example implantable medical device (IMD);

FIG. 2C is a plan view illustrating an example implantable medical device (IMD);

FIG. 2D is a top plan view illustrating an example implantable medical device (IMD);

FIG. 3 is a functional block diagram illustrating an example configuration of the IMD of FIGS. 1 and 2A-2D;

FIG. 4A is a cross-sectional view illustrating an example implantable medical device (IMD) within a delivery cup;

FIG. 4B is a plan view illustrating an example implantable medical device (IMD);

FIG. 5 is an exploded perspective view illustrating an example medical device system;

FIG. 6A is a plan view illustrating an example medical device delivery system for delivering an IMD to a location within a heart;

FIG. 6B is a conceptual view illustrating an example medical device delivery system for delivering an IMD to a location within a heart;

FIG. 7 is a flow diagram illustrating an example technique for using a medical device system; and

FIG. 8 is a conceptual diagram of a map of a patient's heart in a standard segment view of the left ventricle showing various electrode implantation locations for use with the example implantable medical devices (IMDs).

### DETAILED DESCRIPTION

In general, this disclosure describes example medical devices and medical device delivery systems. This disclosure describes implantable medical devices (IMDs) having header and electrode configurations that enable a clinician to target delivery of pacing pulses to selected cardiac tissue, such as triangle of Koch, a coronary sinus ostium, right atrium, or to a left bundle branch, among other places using the IMD and to reach such target sites using a delivery catheter. Although described herein primarily in the context of targeting certain areas of the heart, the medical devices described herein may be used to any target cardiac tissue that is challenging to reach. In some examples, the IMD may have an angled distal end relative to a reference plane. In some examples, the delivery catheter may have an angled distal end relative to a reference plane. In some examples, the IMD and the delivery catheter may each have an angled distal end relative to a reference plane.

The header and electrode configurations of this disclosure may be implemented in IMDs that include a housing coupled to one or more elongated leads, or in relatively compact IMDs that are sufficiently small to be implanted within a chamber of the patient's heart. The electrodes of the IMD may be configured to, upon IMD implantation, sense electrical signals from tissue and/or deliver electrical therapies to the tissue.

Some electrode configuration examples according to this disclosure additionally or alternatively include a plurality of distal electrodes. The distal electrodes may be configured such that their electrically active portions, e.g., distal ends, are spaced apart from one another in the cardiac tissue. In this manner, the likelihood that at least one of the distal electrodes is proximate to or within a target tissue may be increased, and/or multiple target tissues may be sensed or stimulated. In some examples, the distal electrodes may be individually controlled to deliver electrical stimulation, and an IMD may select which one or more of the distal electrodes is used to stimulate target tissue. In some examples, the plurality of distal electrodes extend, e.g., are curved to extend, in different directions from the distal end of the IMD or lead. An example fixation component for the IMD, e.g., to fix the relatively compact IMD housing or lead distal end to cardiac tissue of the heart chamber in which it is implanted, may include a base and a plurality of fixation tines.

In this disclosure, the example systems, devices, and techniques will be described with reference to delivering electrodes of an IMD configured as a cardiac pacemaker to a target site in the heart of a patient. However, it will be understood that example systems, devices, and techniques of the present disclosure are not limited to delivering such IMD electrodes to particular target sites in the heart. For example, the example systems, devices, and techniques described herein may be used to deliver other medical devices, such as sensing devices, neurostimulation device, medical electrical leads, etc. Additionally, the example systems, devices, and techniques described herein may be used to deliver any such IMDs to other locations within the body of the patient. In short, the example systems, devices, and techniques described herein can find useful application in delivery of a wide variety of implantable medical devices for delivery of therapy to a patient or patient sensing.

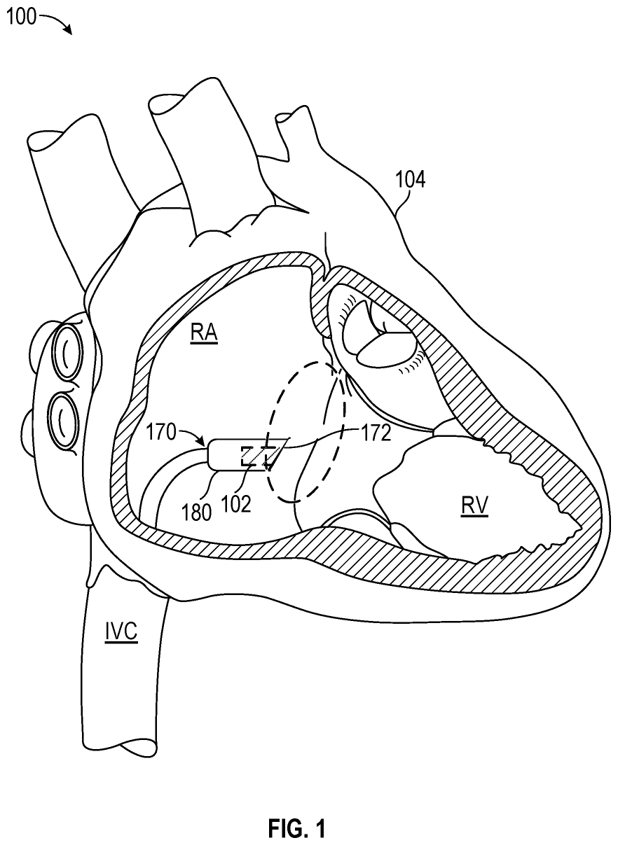

FIG. 1 is a conceptual diagram illustrating a portion of an example medical device system 100 configured to implant a relatively compact implantable medical device 102 (“IMD 102”) at a target implant site. In some examples, as illustrated in FIG. 1, the target implant site 106 may include an appendage or triangle of Koch region of a right atrium (RA) of the heart 104 of a patient. In some examples, as illustrated in FIG. 1, the target implant site 106 may include a coronary sinus ostium (not shown in FIG. 1), or to a left bundle branch (not shown in FIG. 1) on a right septal wall of a patient's heart. In some examples, target implant site may include other portions of heart 104, such as an interventricular septum, or other locations within a body of the patient. Medical device system 100 may include a delivery catheter 170 configured to house and controllably deploy relatively compact IMD 102. In some examples, the delivery catheter 170 includes an angled distal end 172, where the angled distal end is non-orthogonal to a longitudinal axis of the distal end of the catheter. In some examples, a clinician may maneuver medical device system 100 to target implant site. For example, with the IMD loaded within a cup 180 of the delivery catheter 170, the clinician may guide delivery catheter up through the inferior vena cava (IVC) and into the RA of heart 104, and place the angled distal end 172 against the septum of the heart 104. In some examples, other pathways or techniques may be used to guide delivery catheter into other target implant sites within the body of the patient.

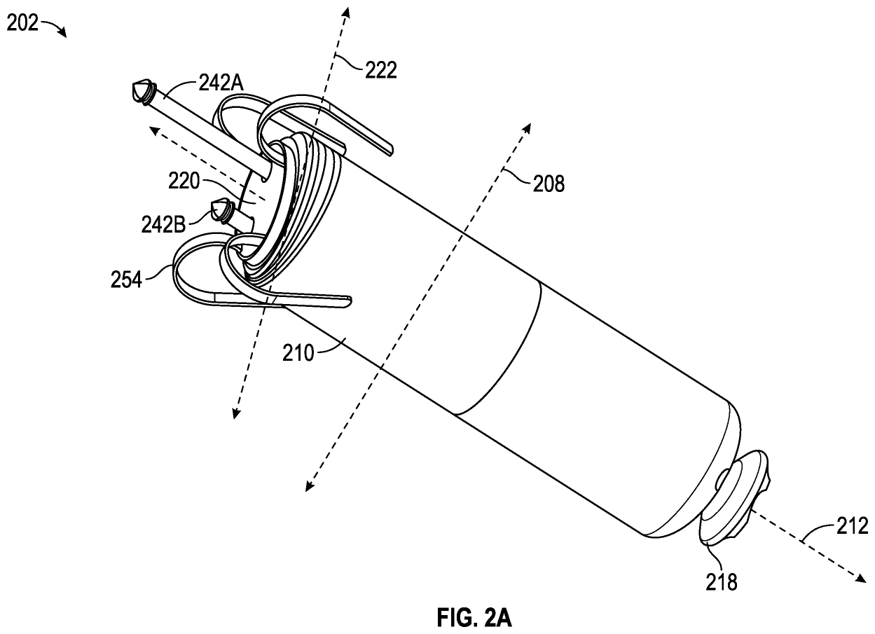

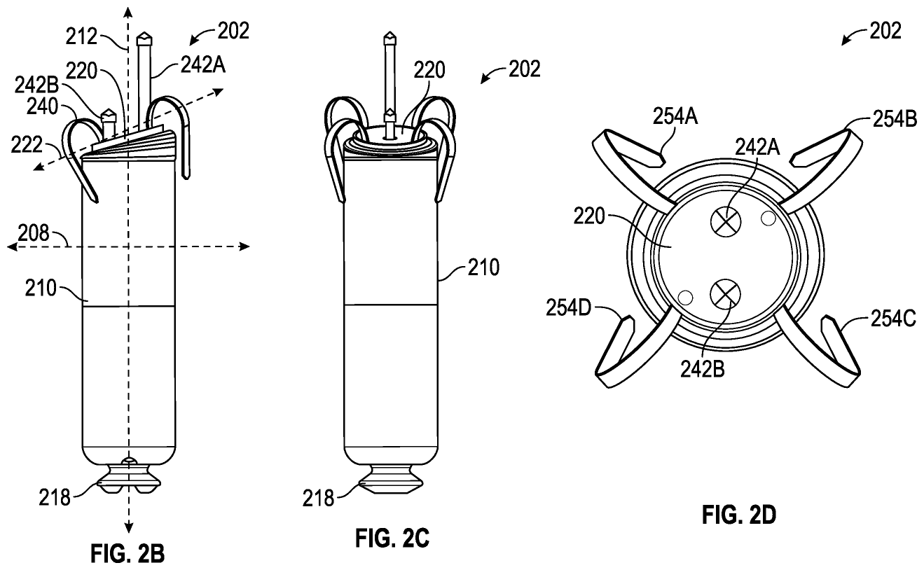

FIGS. 2A-2D illustrates various different views of an example relatively compact IMD 202 including a header and electrode configuration according to the techniques of this disclosure. IMD 202 may be an example of IMD 102 illustrated in FIG. 1. IMD 202 includes housing 210 extending along longitudinal axis 212 from a proximal end to a distal end, and includes a header 220 at the distal end of the housing 210. Housing 210 may be formed from a biocompatible and biostable metal such as titanium. In some examples, housing 210 may include a hermetically sealed housing.

The header 220 defines a header plane 222, and the header plane 222 may be disposed at an angle relative to a reference plane 208, and the reference plane 208 may be perpendicular to the longitudinal axis 212. In some examples, header plane 222 is disposed at a non-orthogonal angle relative to the reference plane 208. In some examples, header plane 222 is disposed at a non-parallel angle relative to the reference plane 208. In some examples, header plane 222 is disposed at an angle of 5-45 degrees from the reference plane 208. In some examples, header plane 222 is disposed at an angle of 15 degrees from the reference plane 208.

IMD 202 may incorporate electronic circuitry within the housing 210, including one or more of sensing circuitry (e.g., for sensing cardiac signals), therapy delivery circuitry (e.g., for generating cardiac pacing pulses), and processing circuitry for controlling the functionality of IMD 202. The circuitry may be disposed within the housing 210, and may be configured to deliver cardiac pacing to the heart via the one or more electrodes 242. In some examples, the one or more electrodes may be an elongated element that extends from the header 220 and may be configured to engage with a septum of a patient. In some examples, the one or more electrodes 242 may include a first electrode 242B extending distally from the header 220 a first length, and a second electrode 242A extending distally from the header 220 a second length, where the second length is greater than the first length. In some examples, the circuitry is configured to deliver atrial pacing therapy (or pacing therapy to a first anatomy) via the first electrode 242B, and ventricular pacing therapy (or pacing therapy to a second anatomy) via the second electrode 242A. In some examples, the first electrode 242B is configured to extend into or contact tissue of a first chamber of the heart, and the second electrode 242A configured to extend into tissue of a second chamber of the heart. In some examples, the one or more electrodes are configured to extend parallel to, and/or obliquely to, the longitudinal axis 212. In one or more examples, the one or more electrodes are configured to pierce the septum of the heart.

In some examples, the one or more electrodes may be linear or nonlinear. Material for the electrodes may include material suitable for pacing and/or sensing tissue of a heart. In some examples, the one or more electrodes may include tines.

IMD 202 may further include a fixation mechanism 240 extending from the header 220, where the fixation mechanism 240 may be configured to be advanced into tissue of a heart of a patient to fix at least a portion of the IMD to the heart. In some examples, the fixation mechanism 240 comprises a plurality of tines 254. As used, herein, a “tine” refers to an elongated element that extends from the housing 210 (e.g., from the distal end thereof), which may be linear or nonlinear. In various examples in accordance with this disclosure, a “tine” may have elastic or super elastic properties, and may, in some cases, be configured to pierce and potentially penetrate into or through target tissue. FIG. 2D illustrates an aerial or end view of IMD 202. In the example shown in FIG. 2D, IMD 202 includes four tines 254 spaced equidistantly about a perimeter of IMD 102, although other number and spacing of tines (e.g., shallow tines, as discussed further herein) are contemplated.

Each of tines 254 may include one or more sections, such as an elastically deformable material preset into one or more curved sections and one or more optional substantially straight sections. Tines 254 may be formed to have a preset deployed shape and may be super elastic, e.g., made of the nickel-titanium alloy Nitinol.

In some examples, IMD 202 includes a retrieval structure 218 fixedly attached to or formed integrally with proximal end of housing 210. Retrieval structure 218 may be configured for temporarily tethering IMD 202 to a delivery catheter or a retrieval catheter, such as delivery catheter.

Tines 254 may be configured to maintain the header 220 and/or electrodes 242 in contact with tissue at a target implant site, e.g., target implant site. A shape of each of tines 254, may be selected to control deployment, tissue fixation, and/or tissue disengagement. For example, the shape of a respective tine may include a number of preset curves on the respective tine, a curvature (e.g., radius) of each preset curve on the respective tine, a length of each preset curve, a length of optional straight sections between preset curves, a width of the respective tine or sections thereof (e.g., one or more tapered portions), a thickness of the respective tine, a number of cutouts along the length of the respective tine, shapes of cutouts, or any combination thereof. In some examples, two or more, or each of the tines 254 may have a different configuration. In some examples, one or more tines, such as a first set of two tines, may have a first length and a first pre-set curve, while one or more other tines, such as a second set of two other tines, may have a second length and a second pre-set curve different than the first length and first pre-set curve. Having the different sets of tines may assist during implant process to implant through an angled delivery cup since two tines will be exposed from the cur first, and then the last two tines will be exposed as the IMD continues to move out of the cup.

Tines 254 may include one or more sections. For example, tines 254 may include an elastically deformable material preset into one or more curved sections and one or more optional substantially straight sections. In some examples, tines 254 may define a ribbon shape configured to deform along a plane that includes longitudinal axis 212 and resist twisting outside of the plane.

Tines 254 may be configured to have a target deflection stiffness and a target deployment stiffness. The target deflection stiffness may include a measure of a resistance to force applied to IMD 202 in a proximal direction when tines 254 are engaged with tissue at target site. In some examples, the target deflection stiffness may be selected to enable tines 254 to deflect a predetermined amount to enable visualization of tines 254 under fluoroscopy.

In some examples, the target deflection stiffness may be within a range from about 0.2 N to about 0.8 N, such as about 0.3 N to about 0.6 N. The deployment stiffness may include a measure of a force applied by tines 254 as tines 254 move from a deformed configuration to an undeformed configuration upon deployment of IMD 202 from distal end 172 of delivery catheter 170 (FIG. 1) such that the free distal end of tines 254 penetrates the atrial or ventricular myocardium. In some examples, the target deployment stiffness may be within a range from about 0.6 N to about 1.2 N.

FIG. 2D illustrates an aerial or end view of IMD 202. In the example shown in FIG. 2D, IMD 202 includes four tines 254 spaced equidistantly about a perimeter of IMD 202, although other number and spacing of tines are contemplated. The illustrated number and arrangement of tines 254 in FIG. 2D is one non-limiting example, and IMD 202 may, in other examples consistent with this disclosure, include a greater number of tines 254 and/or a different position of tines 254 about longitudinal axis 212 of IMD 202. In one non-limiting example, tines 254 may include four deep tines equally distributed circumferentially.

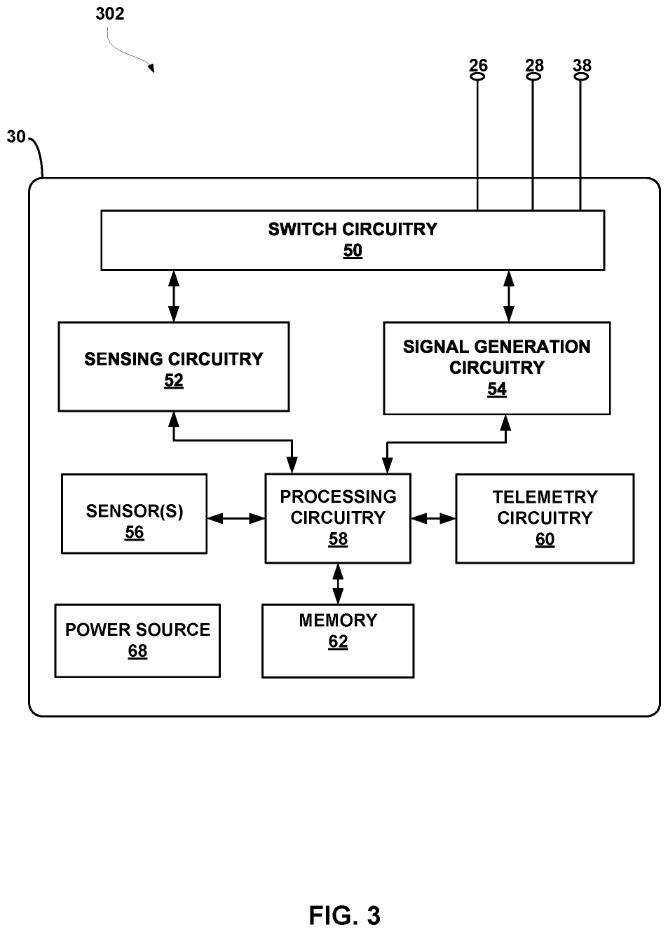

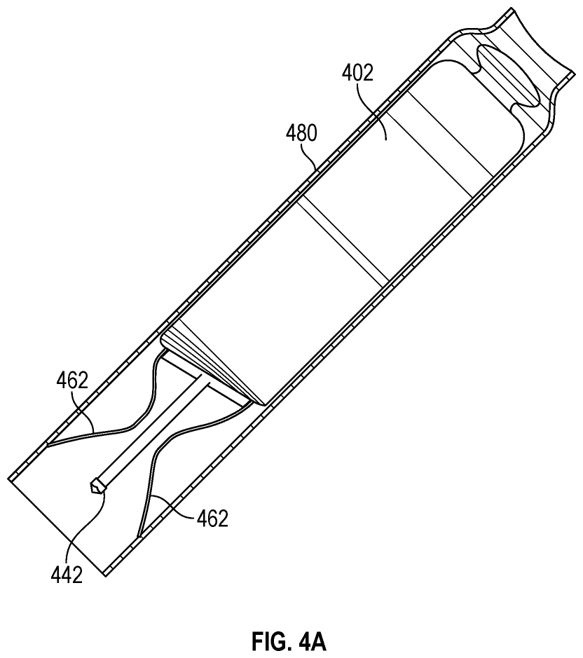

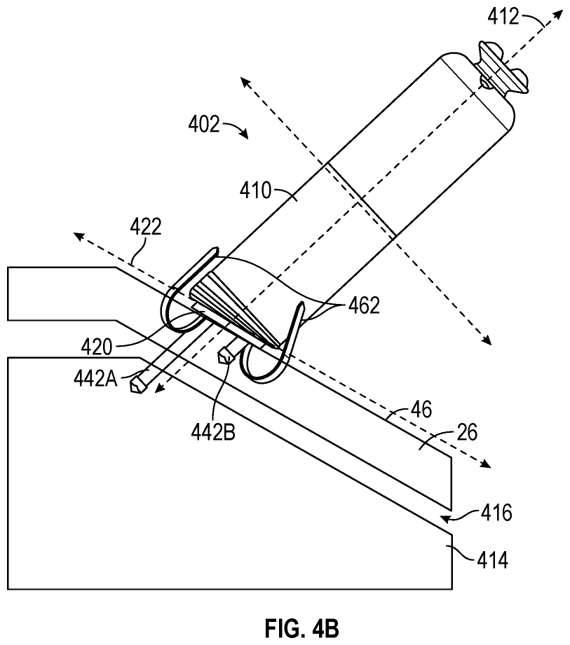

FIG. 3 is a functional block diagram illustrating an example configuration of IMD 302. IMD 302 may be an example of IMD 102 of FIG. 1 and/or IMD 202 of FIGS. 2A-2D, and/or IMD 402 of FIG. 4B. In the example shown in FIG. 3, IMD 302 includes switch circuitry 50, sensing circuitry 52, signal generation circuitry 54, sensor(s) 56, processing circuitry 58, telemetry circuitry 60, memory 62, and power source 68. The various circuitry may be, or include, programmable or fixed function circuitry configured to perform the functions attributed to respective circuitry. Memory 62 may store computer-readable instructions that, when executed by processing circuitry 58, cause IMD 302 to perform various functions. Memory 62 may be a storage device or other non-transitory medium. The components of IMD 302 illustrated in FIG. 3 may be housed within housing 30.

Signal generation circuitry 54 generates electrical stimulation signals, e.g., cardiac pacing pulses. Switch circuitry 50 is coupled to electrodes 26, 28, and 38, may include one or more switch arrays, one or more multiplexers, one or more switches (e.g., a switch matrix or other collection of switches), one or more transistors, or other electrical circuitry. Switch circuitry 50 is configured to direct stimulation signals from signal generation circuitry 54 to a selected combination of electrodes 26, 28, and 38, having selected polarities, e.g., to selectively deliver pacing pulses to the RA, ventricles, conduction system and/or interventricular septum of a patient's heart.

Switch circuitry 50 may also selectively couple sensing circuitry 52 to selected combinations of electrodes 26, 28, and 38, e.g., to selectively sense the electrical activity of either the RA or ventricles of the heart. Sensing circuitry 52 may include filters, amplifiers, analog-to-digital converters, or other circuitry configured to sense cardiac electrical signals via electrodes 26, 28, 38. In some examples, sensing circuitry 52 is configured to detect events, e.g., depolarizations, within the cardiac electrical signals, and provide indications thereof to processing circuitry 58. In this manner, processing circuitry 58 may determine the timing of atrial and ventricular depolarizations, and control the delivery of cardiac pacing, e.g., AV synchronized cardiac pacing, based thereon. Processing circuitry 58 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 58 herein may be embodied as firmware, hardware, software or any combination thereof.

Sensor(s) 56 may include one or more sensing elements that transduce patient physiological activity to an electrical signal to sense values of a respective patient parameter. Sensor(s) 56 may include one or more accelerometers, optical sensors, chemical sensors, temperature sensors, pressure sensors, or any other types of sensors. Sensor(s) 56 may output patient parameter values that may be used as feedback to control sensing and delivery of therapy by IMD 302.

Telemetry circuitry 60 supports wireless communication between IMD 302 and an external programmer (not shown in FIG. 3) or another computing device under the control of processing circuitry 58. Processing circuitry 58 of IMD 302 may receive, as updates to operational parameters from the computing device, and provide collected data, e.g., sensed heart activity or other patient parameters, via telemetry circuitry 60. Telemetry circuitry 60 may accomplish communication by radiofrequency (RF) communication techniques, e.g., via an antenna (not shown).

Power source 68 delivers operating power to various components of IMD 302. Power source 68 may include a rechargeable or non-rechargeable battery and a power generation circuit to produce the operating power. Recharging may be accomplished through proximal inductive interaction between an external charger and an inductive charging coil within IMD 302.

FIG. 4B is a conceptual diagram illustrating IMD 402 implanted at target implant site. As shown in the implanted position, header 420 sits flush with tissue of surface 46, and may be parallel with surface 46, but is disposed at a non-orthogonal angle relative to the longitudinal axis 412 of IMD 402. In some examples, tines 462 may engage the atrial myocardium 26 and electrode 442A pierces tissue at a target implant site, and may advance through the atrial myocardium 26 and central fibrous body 416 to position electrode 442A in the ventricular myocardium 414, and electrode 442B may be positioned in the atrial myocardium 26. In some examples, the shape of tines 462 in a relaxed state may resist movement of the IMD to deform the tines.

In other examples, the distal tips of tines 462 may extend a length from the proximal base that is substantially similar or greater than the length of electrodes 442 when IMD 402 is held in a retracted position within a cup 480, as shown in FIG. 4A. As the IMD 402 is released from the cup 480 and toward the tissue, the distal tips of tines 462 pierce the tissue first in this case, and may act to pull IMD 402 toward the atrial endocardial surface 46 as tines 462 elastically bend or curve back, toward or into the curved position (See FIG. 4B). This pulling force produced by tines 462 may contribute to the longitudinal force that drives the electrodes 442A, 442B into the tissue at target implant site and advances the tip electrodes toward the pacing site. In some examples, the pulling force produced by tines 462 may drive the electrodes 442A, 442B into the heart tissue to a desired depth to deliver pacing, thereby reducing the overall amount of implant force necessary. The angled header 420 is advanced to contact the atrial endocardial surface 46 as the tines 462 are deployed.

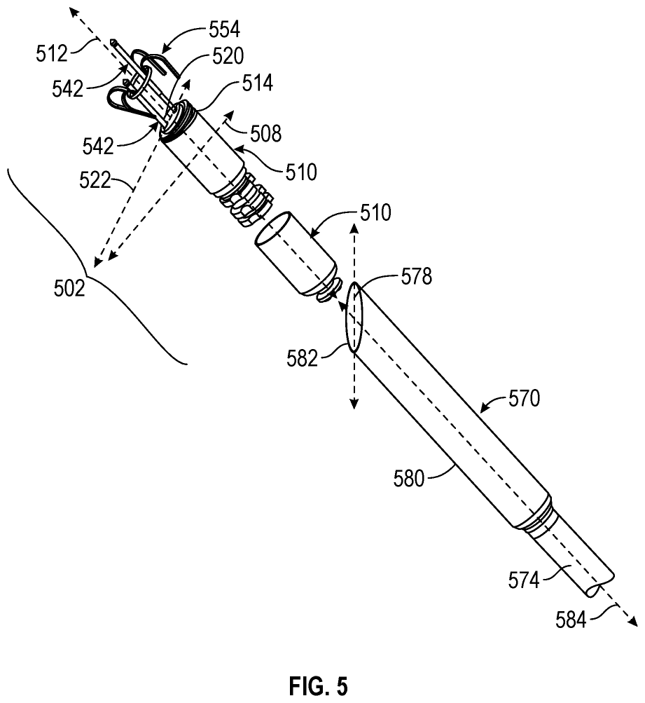

FIG. 5 illustrates an exploded conceptual view of an IMD 502 and a delivery catheter 570. The IMD 502 (which can optionally be similar to any of the other embodiments of the IMD disclosed herein, except as further described) has a housing 510 that may be defined in part by a longitudinal axis 512. The IMD 502 may further include a header 520 disposed at a distal end 514 of the IMD 502 (e.g., the housing 510 thereof). The header 520 may define a header plane 522, and the header plane 522 may be disposed at an angle relative to a reference plane 508. In some examples, the reference plane 508 is orthogonal to the longitudinal axis 512 of IMD 502. In some examples, the header plane 522 is disposed at a non-parallel angle relative to the reference plane 508.

The IMD may further include a fixation mechanism extending from the header 520. An example fixation component for the IMD, e.g., to fix a relatively compact IMD housing to cardiac tissue of the heart chamber in which it is implanted, may include a plurality of fixation tines 554. In some examples, the plurality of tines may include a first set of tines and a second set of tines, the first set of tines having a different length than the second set of tines. In some examples, the plurality of tines is configured to be advanced into the septum (and/or other tissue of the heart) by piercing the septum or other tissue.

Delivery catheter 570 includes a cup 580 which holds the IMD 502 during the implant process, and a body 574 to guide the cup 580 during the implant process. Cup 580 has a distal cup end with an opening 578 to receive the IMD 602. Cup 580 is further defined by a cup end plane 582 and a cup longitudinal axis 584. In one or more examples, the cup end plane 582 is disposed at a cup angle relative to the cup longitudinal axis 584. In some examples, the cup end plane 582 is disposed at a non-orthogonal angle relative to the cup longitudinal axis 584. In some examples, the cup angle is 5-45 degrees relative to the cup longitudinal axis. In some examples, the cup angle is 15 degrees relative to the cup longitudinal axis. In some examples, the cup end plane 582 is disposed at a cup angle relative to the cup longitudinal axis 584 and the opening 578 may have an oval shape. In some examples, an angle of displacement of the cup may match or otherwise similar to an angle of displacement of the header plane.

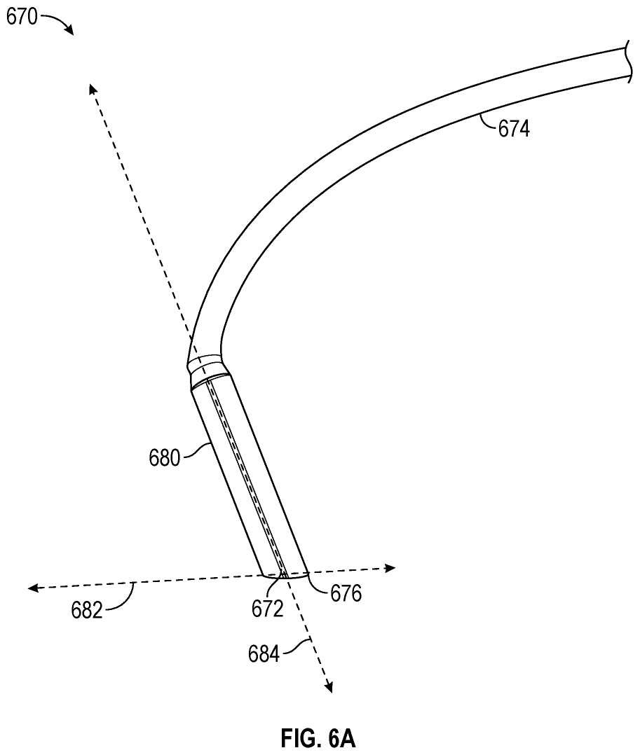

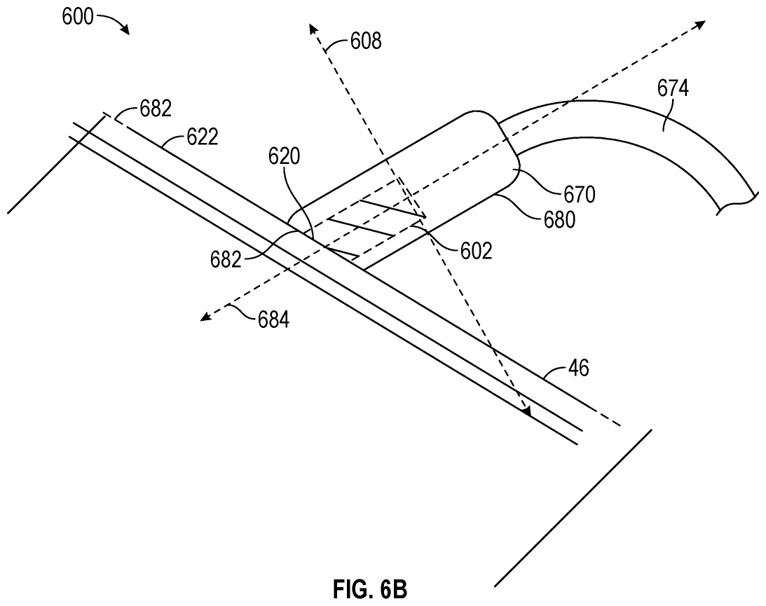

FIGS. 6A and 6B illustrate a delivery catheter 670 which extends to a distal end 672. Delivery catheter 670 includes a cup 680 which holds the IMD 602 (FIG. 6B) during the implant process, and a body 674 to guide the cup 680 during the implant process. Cup 680 has a distal cup end 676 with an opening to receive the IMD 602. Cup 680 is further defined by a cup end plane 682 and a cup longitudinal axis 684. In one or more examples, the cup end plane 682 is disposed at a cup angle relative to the cup longitudinal axis 684. In some examples, the cup end plane 682 is disposed at a non-orthogonal angle relative to the cup longitudinal axis 684. In some examples, the cup angle is 5-45 degrees relative to the cup longitudinal axis. In some examples, the cup angle is 15 degrees relative to the cup longitudinal axis.

In some examples, delivery catheter 670 is a deflectable catheter. For examples, a clinician, while implanting an IMD may have an ability to deflect the catheter, for example curving the catheter. In some examples, the rate of deflection or curving may be predetermined. In some examples, the deflectable catheter is configured to deflect such that the cup end plane is substantially parallel to tissue of the patient when the IMD is delivered to the septum. In some examples, the deflectable catheter is configured to deflect such that the cup end plane is parallel to tissue of the patient when the IMD is delivered to the septum. In some examples, the deflectable catheter is configured to deflect such that the cup end plane is parallel to tissue of the patient when the IMD is delivered to the septum, and the cup longitudinal axis 684 is non-orthogonal with tissue of the patient when the IMD is delivered to the septum.

FIG. 6B illustrates the distal end of cup 680 of delivery catheter 670 pressed against tissue, such as an atrial endocardial surface 46 at the implant site of a heart. The delivery catheter 670 includes an IMD 602 disposed within the cup 680. The IMD 602 includes a header 620 having a header plane 622, and the header plane 622 is angled relative to the longitudinal axis 684. In some examples, the cup end plane 682 is parallel with the header plane 622.

When a clinician is satisfied with the positioning of cup 680 with respect to tissue, e.g., that the cup end plane 682 of cup 680 is generally parallel to a plane defined by surface 46, and/or that cup 682 is pressed sufficiently against/into surface 46 such that fixation members such as tines 254 (FIGS. 2A-2D) of IMD 602 will deploy into the tissue, the clinician may advance IMD 602 towards the tissue. Fixation mechanism 240 (FIGS. 2A-2D) may be configured to embed into tissue of surface 46, and in some cases pull IMD 602 through a distal opening of cup 680, when advanced through the distal opening. While in some examples IMD 602 is shown having fixation members that includes a plurality of tine structures, it should be understood that IMD 602 may include any other suitable fixation structure or structures.

During delivery of IMD 602 to the treatment site via delivery catheter 670, a clinician may advance cup 680 into contact with tissue of surface 46 prior to engaging fixation members with tissue of surface 46. The clinician then may determine whether cup 680 and IMD 602 are properly positioned at the implant site prior to engaging fixation members with the tissue of surface 46. In some examples, the clinician may determine whether cup 680 and IMD 602 are properly positioned relative to heart based on an impedance or other electrical signal sensed via an electrical path including IMD 602. In addition to the IMD 602, another electrode of the electrical path may be a reference electrode attached to the patient, or inside the patient but located outside of cup 680. In some examples, relatively higher impedance may be indicative of cup 680 being positioned flush against, and with adequate depth in, tissue of surface 46, which may be desirable for proper fixation.

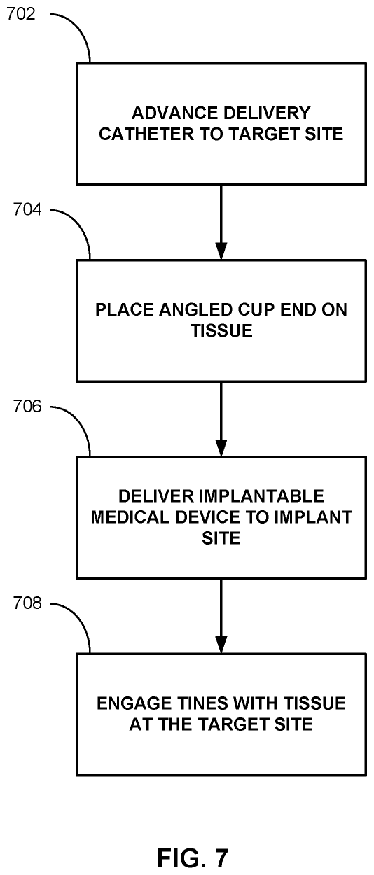

FIG. 7 is a flow diagram illustrating an example technique for implanting an implantable medical device according to aspects of this disclosure. The example technique of FIG. 7 is described in the context of IMD 202 (FIGS. 2A-2D) and catheter 670 of FIGS. 6A—6B but may be applicable to other embodiments of the IMD and/or catheter disclosed herein, or to other medical devices. According to the example of FIG. 6B, a delivery catheter 670 or sheath used to guide the IMD 602 to a target implantation site in the heart is advanced to the target implantation site (702). In some examples, the catheter is advanced to the target site with IMD 602 disposed in its cup 680.

In some examples, cup 680 is manipulated, for example by the clinician deflecting the delivery catheter 170 (FIG. 1) to place the angled cup end plane 682 of the cup 680 on tissue of the target site (704). During delivery of IMD 602 to the treatment site via delivery catheter 670, a clinician may advance cup 680 into contact with tissue of surface 46 prior to engaging fixation members with tissue of surface 46. The clinician then may determine whether cup 680 and IMD 602 are properly positioned at the implant site prior to engaging fixation members with the tissue of surface 46.

Once the delivery catheter is advanced to the target implantation site, IMD 602 may be delivered to the implant site (706). In some examples, delivering the IMD to an implant site with the catheter comprises delivering the IMD to a triangle of Koch. In some examples, delivering the IMD to an implant site with the catheter comprises delivering the IMD to a coronary sinus ostium. In some examples, delivering the IMD to an implant site with the catheter comprises delivering the IMD to a left bundle branch on a right septal wall.

In some examples, the IMD 602 may be advanced out of cup 680 towards tissue of the implant site. In some examples, IMD 602 may be delivered with IMD 602 already advanced to a distal end of the delivery catheter, while in others IMD 602 may be retracted from the distal end of the delivery catheter, e.g., to protect patient tissue from electrode 242 during navigation to the target implantation site.

According to the example of FIG. 7, IMD is advanced out of the cup 680, and tines 254 are advanced out of the distal end of the delivery catheter through the tissue at the target implantation site and into to deeper tissue, such as tissue in one or more chambers of the heart, or other conduction system tissues of the heart (708). In some examples, the tines 254 may be generally straight within the cup 680, and when released from the cup 680 curl into the tissue and engage the tissue. As IMD is advanced out of the cup 680 and the tines engage the tissue, electrodes 242 also are embedded into tissue.

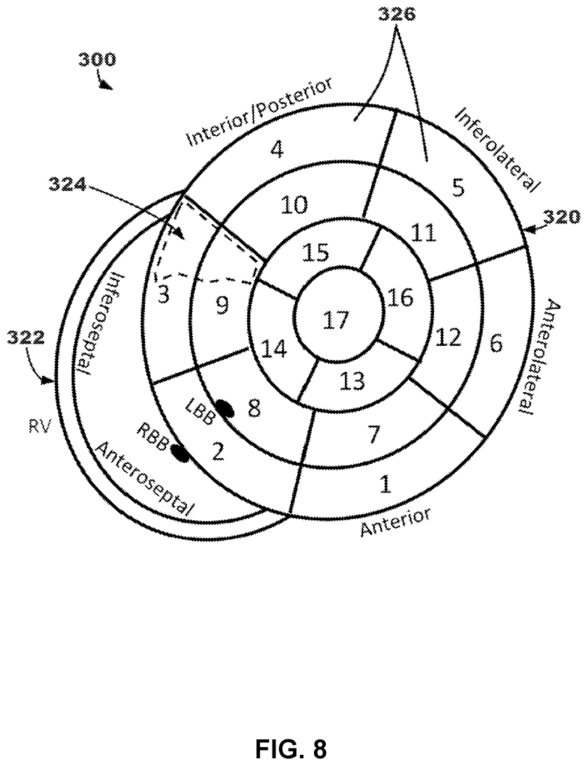

FIG. 8 is a two-dimensional (2D) ventricular map 300 of a patient's heart (e.g., a top-down view) showing the left ventricle 320 in a standard 17 segment view and the right ventricle 322. The map 300 includes a plurality of areas 326 corresponding to different regions of a human heart. As illustrated, the areas 326 are numerically labeled 1-17 (which, e.g., correspond to a standard 17 segment model of a human heart, correspond to 17 segments of the left ventricle of a human heart, etc.). Areas 326 of the map 300 may include basal anterior area 1, basal anteroseptal area 2, basal inferoseptal area 3, basal inferior area 4, basal inferolateral area 5, basal anterolateral area 6, mid-anterior area 7, mid-anteroseptal area 8, mid-inferoseptal area 9, mid-inferior area 10, mid-inferolateral area 11, mid-anterolateral area 12, apical anterior area 13, apical septal area 14, apical inferior area 15, apical lateral area 16, and apex area 17. The inferoseptal and anteroseptal areas of the right ventricle 322 are also illustrated, as well as the right bunch branch (RBB) and left bundle branch (LBB).

In some embodiments, any of the tissue-piercing electrodes of the present disclosure may be implanted in the basal and/or septal region of the left ventricular myocardium of the patient's heart. In particular, the tissue-piercing electrode may be implanted from the triangle of Koch region of the right atrium through the right atrial endocardium and central fibrous body.

Once implanted, the tissue-piercing electrode may be positioned in the target implant region, such as the basal and/or septal region of the left ventricular myocardium. With reference to map 300, the basal region includes one or more of the basal anterior area 1, basal anteroseptal area 2, basal inferoseptal area 3, basal inferior area 4, mid-anterior area 7, mid-anteroseptal area 8, mid-inferoseptal area 9, and mid-inferior area 10. With reference to map 300, the septal region includes one or more of the basal anteroseptal area 2, basal anteroseptal area 3, mid-anteroseptal area 8, mid-inferoseptal area 9, and apical septal area 14.

In some embodiments, the tissue-piercing electrode may be positioned in the basal septal region of the left ventricular myocardium when implanted. The basal septal region may include one or more of the basal anteroseptal area 2, basal inferoseptal area 3, mid-anteroseptal area 8, and mid-inferoseptal area 9.

In some embodiments, the tissue-piercing electrode may be positioned in the high inferior/posterior basal septal region of the left ventricular myocardium when implanted. The high inferior/posterior basal septal region of the left ventricular myocardium may include a portion of at least one of the basal inferoseptal area 3 and mid-inferoseptal area 9. For example, the high inferior/posterior basal septal region may include region 324 illustrated generally as a dashed-line boundary. As shown, the dashed line boundary represents an approximation of about where the high inferior/posterior basal septal region and may take somewhat different shape or size depending on the particular application. Without being bound by any particular theory, intraventricular synchronous pacing and/or activation may result from stimulating the high septal ventricular myocardium due to functional electrical coupling between the subendocardial Purkinje fibers and the ventricular myocardium.

Example 1: An implantable medical device (IMD) includes a housing defined in part by a longitudinal axis; a header at a distal end of the housing, the header defining a header plane, the header plane disposed at an angle relative to a reference plane, where the reference plane is perpendicular to the longitudinal axis; a fixation mechanism extending from the header, the fixation mechanism configured to be advanced into tissue of a heart of a patient to fix at least a portion of the IMD to the heart; one or more electrodes that extend from the header and are configured to engage with the septum; and circuitry within the housing, wherein circuitry is configured to deliver cardiac pacing to the heart via the one or more electrodes.

Example 2: The IMD of example 1, wherein the header plane is disposed at an angle of 5-45 degrees from the reference plane.

Example 3: The IMD of example 2, wherein the angle is 15 degrees.

Example 4: The IMD of any one or more of examples 1-3, wherein the one or more electrodes comprise: a first electrode extending distally from the header a first length; and a second electrode extending distally from the header a second length, wherein the second length is greater than the first length.

Example 5: The IMD of example 4, wherein the circuitry is configured to deliver atrial pacing therapy via the first electrode, and ventricular pacing therapy via the second electrode.

Example 6: The IMD of example 4 or 5, wherein the first electrode is configured to extend into tissue of a first chamber of the heart, and the second electrode configured to extend into tissue of a second chamber of the heart.

Example 7: The IMD of any one or more of examples 1-6, wherein the one or more electrodes are configured to extend parallel to the longitudinal axis.

Example 8: The IMD of any one or more of examples 1-7, wherein the one or more electrodes are configured to pierce the septum.

Example 9: The IMD of any one or more of examples 1-8, wherein the fixation mechanism comprises a plurality of tines.

Example 10: The IMD of example 9, wherein the plurality of tines includes a first set of tines and a second set of tines, the first set of tines having a different length than the second set of tines.

Example 11: The IMD of example 9 or 10, wherein the plurality of tines is configured to be advanced into the septum by piercing the septum.

Example 12: A medical device system includes an implantable medical device (IMD) having a housing defined in part by a longitudinal axis, a header at a distal end of the housing, the header defining a header plane, the header plane disposed at an angle relative to a reference plane, where the reference plane is perpendicular to the longitudinal axis, a fixation mechanism extending from the header, the fixation mechanism configured to be advanced into tissue of a heart of a patient to fix at least a portion of the IMD to the heart, one or more electrodes that extend from the header and are configured to engage with the septum, and circuitry within the housing, wherein circuitry is configured to deliver cardiac pacing to the heart via the one or more electrodes; and a catheter extending to a distal end, the catheter having a cup at the distal end of the catheter, the cup having a distal cup end, the cup end defined by a cup end plane and a cup longitudinal axis, and the IMD is configured to be disposed within the cup while the IMD is delivered to the septum within the patient.

Example 13: The medical device system of example 12, wherein the cup end plane is disposed at a cup angle relative to the cup longitudinal axis.

Example 14: The medical device system of example 13, wherein the cup angle is 5-45 degrees relative to the cup longitudinal axis.

Example 15: The medical device system of example 14, wherein the cup angle is 15 degrees.

Example 16: The medical device system of any one or more of examples 13-15, wherein the cup end plane is parallel with the header plane.

Example 17: The medical device system of any one or more of examples 12-15, wherein the catheter is a deflectable catheter.

Example 18: The medical device system of example 17, wherein the deflectable catheter is configured to deflect such that the cup end plane is parallel to tissue of the patient when the IMD is delivered to the septum.

Example 19: The medical device system of any one or more of examples 12-18, wherein the header plane is disposed at an angle of 5-45 degrees from the reference plane.

Example 20: The medical device system of example 19, wherein the angle is 15 degrees.

Example 21: The medical device system of any one or more of examples 12-20, wherein the one or more electrodes comprise: a first electrode extending distally from the header a first length; and a second electrode extending distally from the header a second length, wherein the second length is greater than the first length.

Example 22: The medical device system of example 21, wherein the circuitry is configured to deliver atrial pacing therapy via the first electrode, and ventricular pacing therapy via the second electrode.

Example 23: The medical device system of example 21, wherein the first electrode is configured to extend into tissue of a first chamber of the heart, and the second electrode configured to extend into tissue of a second chamber of the heart.

Example 24: The medical device system of any one or more of examples 12-23, wherein the one or more electrodes are configured to extend parallel to the longitudinal axis.

Example 25: The medical device system of any one or more of examples 12-24, wherein the one or more electrodes are configured to pierce the septum.

Example 26: The medical device system of any one or more of examples 12-25, wherein the fixation mechanism comprises a plurality of tines.

Example 27: The medical device system of example 26, wherein the plurality of tines includes a first set of tines and a second set of tines, the first set of tines having a different length than the second set of tines.

Example 28: The medical device system of example 26 or 27, wherein the plurality of tines is configured to be advanced into the septum by piercing the septum.

Example 29: A method includes advancing a medical device system within a patient, the medical device system comprising a catheter extending to a distal end, the catheter having a cup at the distal end of the catheter, the cup having a distal cup end, the cup end defined by a cup end plane and a cup longitudinal axis, the cup end plane is disposed at a cup angle relative to the cup longitudinal axis, an implantable medical device (IMD) disposed within the cup, the (IMD) having a housing defined in part by a longitudinal axis, a header at a distal end of the housing, the header defining a header plane, the header plane disposed at an angle relative to a reference plane, where the reference plane is perpendicular to the longitudinal axis, a fixation mechanism extending from the header, the fixation mechanism configured to be advanced into tissue of a heart of a patient to fix at least a portion of the IMD to the heart, one or more electrodes that extend from the header and are configured to engage with the septum, and circuitry within the housing, wherein circuitry is configured to deliver cardiac pacing to the heart via the one or more electrodes; delivering the IMD to an implant site with the catheter and placing the cup end plane on the tissue where the cup longitudinal axis is non-orthogonal relative to a tissue plane; advancing the IMD from the cup; and engaging the plurality of tines with the tissue.

Example 30: The method of example 29, further comprising engaging the one or more electrodes with the tissue, where the longitudinal axis of the housing is non-orthogonal relative to the tissue plane.

Example 31: The method of example 29 or 30, wherein delivering the IMD to an implant site with the catheter and placing the cup end plane on the tissue includes deflecting the distal end of the catheter.

Example 32: The method of any one or more of examples 29-31, wherein delivering the IMD to an implant site with the catheter comprises delivering the IMD to a triangle of Koch.

Example 33: The method of any one or more of examples 29-32, wherein delivering the IMD to an implant site with the catheter comprises delivering the IMD to a coronary sinus ostium.

Example 34: The method of any one or more of examples 29-33, wherein delivering the IMD to an implant site with the catheter comprises delivering the IMD to a left bundle branch on a right septal wall.

Example 35: An implantable medical device (IMD) comprising: an IMD body extending along a longitudinal axis of the IMD; a distal end portion forming a distal end of the IMD body, the distal end portion oriented on a distal end plane, the distal end plane disposed at an angle relative to a reference plane, wherein the reference plane is orthogonal to the longitudinal axis; a fixation mechanism extending from the distal end of the IMD body, the fixation mechanism configured to be advanced into tissue of a heart of a patient to fix at least a portion of the IMD to the heart; one or more electrodes that extend from the distal end portion and are configured to engage with tissue of the heart; and circuitry within the IMD body, wherein the circuitry is configured to deliver cardiac pacing to the heart via the one or more electrodes.

Example 36: The IMD of example 35, wherein the one or more electrodes comprise: a first electrode extending distally from the distal end plane a first length; and a second electrode extending distally from the distal end plane a second length, wherein the second length is greater than the first length.

Example 37: The IMD of example 35 or 36, wherein the circuitry is configured to deliver atrial pacing therapy via the first electrode, and ventricular pacing therapy via the second electrode.

Example 38: The IMD of any one or more of examples 35-37, wherein the one or more electrodes are configured to extend parallel to the longitudinal axis.

Example 39: The IMD of any one or more of examples 35-38, wherein the one or more electrodes are configured to pierce a septum of the heart.

Example 40: The IMD of any one or more of examples 35-39, wherein the fixation mechanism comprises a plurality of tines.

Example 41: The IMD of example 40, wherein the plurality of tines includes a first set of tines and a second set of tines, the first set of tines having a different length than the second set of tines.

Example 42: An implantable medical device (IMD) system comprising: an IMD body extending along a longitudinal axis of the IMD; a distal end portion forming a distal end of the IMD body, the distal end portion oriented on a distal end plane, the distal end plane disposed at an angle relative to a reference plane, wherein the reference plane is orthogonal to the longitudinal axis; a fixation mechanism extending from the distal end of the IMD body, the fixation mechanism configured to be advanced into tissue of a heart of a patient to fix at least a portion of the IMD to the heart; one or more electrodes that extend from the distal end portion and are configured to engage with tissue of the heart; and circuitry within the IMD body, wherein the circuitry is configured to deliver cardiac pacing to the heart via the one or more electrodes; a catheter extending to a distal end, the catheter having a cup at the distal end of the catheter, the cup having a distal cup end, the cup end defined by a cup end plane and a cup longitudinal axis, and the IMD is configured to be disposed within the cup while the IMD is delivered to the septum within the patient.

Example 43: The medical device system of example 42, wherein the cup end plane is disposed at a cup angle relative to the cup longitudinal axis.

Example 44: The medical device system of example 43, wherein the cup angle is 5-45 degrees relative to the cup longitudinal axis.

Various aspects of the disclosure have been described. These and other aspects are within the scope of the following claims.

What is claimed is:

1. An implantable medical device (IMD) comprising:
a housing defining a longitudinal axis;
a header at a distal end of the housing, the header defining a header plane, the header plane disposed at an angle relative to a reference plane, where the reference plane is perpendicular to the longitudinal axis;
a fixation mechanism extending from the header, the fixation mechanism configured to be advanced into tissue of a heart of a patient to fix at least a portion of the IMD to the heart;
one or more electrodes that extend from the header and are configured to engage with heart tissue; and
circuitry within the housing, wherein the circuitry is configured to deliver cardiac pacing to the heart via the one or more electrodes.

2. The IMD of claim 1, wherein the angle is 5-45 degrees from the reference plane.

3. The IMD of claim 2, wherein the angle is 15 degrees.

4. The IMD of claim 1, wherein the one or more electrodes comprise:
a first electrode extending distally from the header a first length; and
a second electrode extending distally from the header a second length, wherein the second length is greater than the first length.

5. The IMD of claim 4, wherein the circuitry is configured to deliver atrial pacing therapy via the first electrode, and ventricular pacing therapy via the second electrode.

6. The IMD of claim 4, wherein the first electrode is configured to extend into tissue of a first chamber of the heart, and the second electrode configured to extend into tissue of a second chamber of the heart.

7. The IMD of claim 1, wherein the one or more electrodes are configured to extend parallel to the longitudinal axis.

8. The IMD of claim 1, wherein the one or more electrodes are configured to pierce a septum of the heart.

9. The IMD of claim 1, wherein the fixation mechanism comprises a plurality of tines.

10. The IMD of claim 9, wherein the plurality of tines includes a first set of tines and a second set of tines, the first set of tines having a different length than the second set of tines.

11. The IMD of claim 9, wherein the plurality of tines is configured to be advanced into the septum by piercing the septum.

12. A medical device system comprising:
an implantable medical device (IMD) having a housing defining a longitudinal axis, a header at a distal end of the housing, the header defining a header plane, the header plane disposed at an angle relative to a reference plane, where the reference plane is perpendicular to the longitudinal axis, a fixation mechanism extending from the header, the fixation mechanism configured to be advanced into tissue of a heart of a patient to fix at least a portion of the IMD to the heart, one or more electrodes that extend from the header and are configured to engage with heart tissue, and circuitry within the housing, wherein the circuitry is configured to deliver cardiac pacing to the heart via the one or more electrodes; and
a catheter extending to a distal end, the catheter having a cup at the distal end of the catheter, the cup having a distal cup end, the distal cup end defined by a cup end plane and a cup longitudinal axis, wherein the IMD is configured to be disposed within the cup while the IMD is delivered to a septum within the patient.

13. The medical device system of claim 12, wherein the cup end plane is disposed at a cup angle relative to the cup longitudinal axis.

14. The medical device system of claim 13, wherein the cup angle is 5-45 degrees relative to the cup longitudinal axis.

15. The medical device system of claim 14, wherein the cup angle is 15 degrees.

16. The medical device system of claim 13, wherein the cup end plane is parallel with the header plane.

17. The medical device system of claim 12, wherein the catheter is a deflectable catheter.

18. The medical device system of claim 17, wherein the deflectable catheter is configured to deflect such that the cup end plane is parallel to tissue of the patient when the IMD is delivered to the septum.

19. The medical device system of claim 12, wherein the angle is 5-45 degrees from the reference plane.

20. The medical device system of claim 19, wherein the angle is 15 degrees.

21. The medical device system of claim 12, wherein the one or more electrodes comprise:
a first electrode extending distally from the header a first length; and
a second electrode extending distally from the header a second length, wherein the second length is greater than the first length.

22. The medical device system of claim 21, wherein the circuitry is configured to deliver atrial pacing therapy via the first electrode, and ventricular pacing therapy via the second electrode.

23. The medical device system of claim 21, wherein the first electrode is configured to extend into tissue of a first chamber of the heart, and the second electrode configured to extend into tissue of a second chamber of the heart.

24. A method comprising:
advancing a medical device system within a patient, the medical device system comprising a catheter extending to a distal end, the catheter having a cup at the distal end of the catheter, the cup having a distal cup end, the cup end defined by a cup end plane and a cup longitudinal axis, wherein the cup end plane is disposed at a cup angle relative to the cup longitudinal axis, an implantable medical device (IMD) disposed within the cup, the IMD comprising a housing having a header, the header having one or more electrodes, the housing defined in part by a longitudinal axis, the header is defined by a header plane, the header plane disposed at an angle relative to a reference plane, where the reference plane is perpendicular to the longitudinal axis, the IMD comprising one or more electrodes configured to be advanced into heart tissue of a patient, wherein the one or more electrodes are configured to deliver cardiac pacing to the heart tissue;
delivering the IMD to an implant site with the catheter and placing the cup end plane on the tissue where the cup longitudinal axis is non-orthogonal relative to a tissue plane;
advancing the IMD from the cup; and
engaging the one or more electrodes with the tissue.

25. The method of claim 24, where the longitudinal axis of the housing is non-orthogonal relative to the tissue plane when engaging the one or more electrodes with the tissue.

26. The method of claim 24, wherein delivering the IMD to the implant site with the catheter and placing the cup end plane on the tissue includes deflecting the distal end of the catheter.

27. The method of claim 24, wherein delivering the IMD to the implant site with the catheter comprises delivering the IMD to a triangle of Koch.

28. The method of claim 24, wherein delivering the IMD to the implant site with the catheter comprises delivering the IMD to a coronary sinus ostium.

29. The method of claim 24, wherein delivering the IMD to the implant site with the catheter comprises delivering the IMD to a right septal wall such that at least one of the one or more electrodes is proximate to a left bundle branch.
